(19) United States
(12) Patent Application Publication
(10) Pub. No.: US 2008/0287566 A1
(43) Pub. Date: Nov. 20, 2008

(54) Epoxy based oil free root canal sealer

(75) Inventors: Barry L. Musikant, New York, NY (US); Allan S. Deutsch, New York, NY (US); Brian Rasimick, New York, NY (US); Quan Gu, New Milford, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Essential Dental Systems, Inc., South Hackensack, NJ (US)

(21) Appl. No.: 11/888,329

(22) Filed: Jul. 31, 2007

Related U.S. Application Data
(63) Continuation-in-part of application No. 11/528,096, filed on Sep. 27, 2006.
(60) Provisional application No. 60/721,309, filed on Sep. 28, 2005.

(51) Int. Cl. A61K 6/00; C08G 59/02

(52) U.S. Cl. 523/118; 528/418

(57) ABSTRACT

Epoxy polymers for dental use including the reaction product of a diepoxide oligomer and a bridged dipolyamine are described. The epoxy polymers include the reaction product of diepoxide oligomers comprised of bisphenol A diepoxide oligomers and/or bisphenol F diepoxide oligomers and bridged dipolyamine monomers having two polyamine regions and a hydrocarbon region of at least 28 carbon atoms.

### CROSS-REFERENCE TO RELATED APPLICATIONS

The present application is a continuation-in-part of U.S. application Ser. No. 11/528,096, filed Sep. 27, 2006, which claims benefit of U.S. Provisional Application Ser. No. 60/721,309, filed Sep. 28, 2005, the disclosures of which are hereby incorporated by reference herein.

### BACKGROUND OF THE INVENTION

Pulp, infected dentin and other materials in the root canal chamber are removed when treating an infected root canal. Once a dentist has removed diseased and soft tissue from the root canal, the chamber must be filled. The purpose of filling the root canal is to seal the area and to eliminate the possibility of bacterial infection in the root canal chamber. Filling and sealing the chamber also prohibits material from draining into the canal and provides a base that may be drilled out later for placement of one or more posts to which may be mounted a crown or other restorative appliance.

The desirable properties for a root canal filling material include ease of removal from the canal if further work is needed, ease of handling with ample working time, minimal shrinkage or change of form after insertion, ability of the material to conform and adapt to the various shapes and contours of an individual's root canals, lack of irritation to tissues, inertness and insolubility in oral fluids. In addition to the foregoing, a desirable root canal filling material is preferably radiopaque and provides antibacterial properties.

Endodontic cones made of gutta percha are currently the root canal filling material of choice because, with careful manipulation, gutta percha can fulfill many of the above requirements. These gutta percha endodontic cones, often called “points”, are typically composed of a mixture of polymer (usually trans isopropene), zinc oxide as a filler, one or more barium- or strontium-containing compounds as a filler and radiopacifier, waxes or resins, pigments and plasticizers.

One difficulty with using gutta percha has been that it does not readily bond to and seal against the tooth tissue.

The most common dental composition or sealer used to bond to the endodontic cone and seal a root canal is a zinc oxide-eugenol made from a powder/liquid configuration. The powder is composed of zinc oxide and other additives such as a radiopacifier and colloidal silica, and the liquid is composed of eugenol and other resinous materials and additives such as a plasticizer. When the powder and liquid are mixed, the components undergo a setting reaction and the initially mixed, pasty material slowly becomes a firm, semi-solid material. The sealer is used to fill any gaps between the gutta percha point and the tissue as well as in the canal branches. This zinc oxide dental composition is hydrophobic, and while it bonds well to the gutta percha, its boding to the hydrophilic canal tissue is often poor.

Some resins have better bonding properties to tooth tissue, but show poor bonding with the hydrophobic gutta percha. Many resin sealants in gel form contain silicone oil or mineral oil which decreases the bonding strength and durability of the bond between the tooth tissue and the dental composition and between the gutta percha and the dental composition. The result may be a root canal chamber that is not completely sealed, increasing the chance of irritation or bacterial contamination, leading to infection and possible failure of the root canal procedure. The presence of silicone oil or mineral oil also potentially decreases the shelf-life of the product as the oil separates out over time. In addition to the fact that the gutta percha filling material is often difficult to bond to, the bond is often compromised due to insufficient coating of the sealer on the gutta percha point. Additionally, many sealants require measuring and mixing the components on a mixing pad before application. This can lead to undesirably variations in compositions from procedure to procedure and, inconvenient to use.

For at least the foregoing reasons, there is a need for a root canal filling materials and methods that provide better bonding and seal between the tooth tissue, a dental composition sealant resin, and the gutta percha cone, giving better protection against irritation and infection than those currently in use, is convenient to use.

### SUMMARY OF THE INVENTION

The present invention includes an epoxy polymer for dental use comprising the reaction product of a diepoxide oligomer comprised of a mixture of bisphenol A diepoxide oligomers of the structure

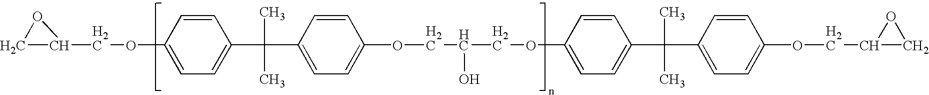

where n ranges from between 0 and 0.5, and/or bisphenol F diepoxide oligomers of the structure

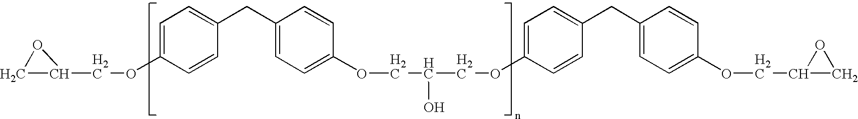

where n ranges from between 0 and 0.5, and mixtures thereof, reacted with bridged dipolyamine monomers wherein said bridged dipolyamine monomers have two polyamine regions and a hydrocarbon region of at least 28 carbon atoms. These formulations can include inorganic fillers which provide radiopacity and/or fumed silica as described herein. Preferably, however, they are substantially free of silicone or mineral oil.

The present invention also includes a dental composition, comprising an epoxy polymer comprising a plurality of amide monomers present in an amount of about 5 to 70 percent by weight of the composition; a plurality of diepoxide monomers present in an amount of about 5 to 70 percent by weight of the composition; at least one fumed silica present in an amount of about 1 to 10 percent by weight of the composition; and at least one inorganic filler which provides radiopacity above 3 mm Al/mm. In a particularly preferred embodiment, the dental composition of the present invention contains substantially no silicone or mineral oil.

In one embodiment, the fumed silica is preferably hydrophobic. In one preferred aspect, the epoxy polymer in this embodiment is a reaction product of mixed diepoxide oligomers containing bisphenols and bridged dipolyamine monomers as described herein.

The present invention also contemplates a root canal sealing dental filling system which includes the dental compositions of the present invention including those just described above. In this system, however, the amide and diepoxide monomers are kept separate, preferably in different portions of at least one dispensing container until they are mixed and dispensed, not necessarily in that order. Each of the other ingredients which are or may be present, such as the fumed silica, the filler, colorant or the like are housed in the same or different containers, preferably the same container. More preferably, they are mixed with and dissolved, dispersed, or suspended with one or the other of the monomers.

More specifically, in accordance with the preferred embodiment, there is provided a dental filling system including a composition comprising a first component comprising a plurality of polymerizable organic amide monomers; and

a second component which can react with the first component to form a polymer, the second component comprising a plurality of polymerizable organic diepoxide monomers. At least one of the first component or the second component further comprises at least one fumed silica. The system also includes, in one embodiment, a dual-chamber dispensing container for holding a dental filling composition and having a first component in a first chamber and a second component in a second chamber, each of the chambers having at least one orifice through which the first component may be removed from the first chamber and the second component can be removed from the second chamber.

In one further embodiment, the dual-chamber device is capable of delivering the first component and the second component simultaneously in any desired ratio and preferably in about a one to one ratio based on the weight, volume or number of molecules of each of the first component and the second component. In another preferred embodiment, the dual-chamber device is a syringe. In another embodiment, the dual-chamber device comprises an automixer to facilitate mixing of the first component and the second component or a mixing chamber wherein the first component and the second component may be mixed before delivery.

The present invention also provides a pre-coated endodontic cone for use in a root canal procedure. The pre-coated endodontic cone comprises an endodontic cone that is sized and shaped so as to be at least partially insertable into an exposed root canal of a tooth; and an epoxy dental polymer comprising a plurality of amide monomers, a plurality of diepoxide monomers and at least one fumed silica is coated on at least one surface of at least a portion of the endodontic cone as described above. In a preferred embodiment, the pre-coated endodontic cone is made of a material selected from gutta percha or polymer and the dental polymer is substantially well distributed over an area of 1 mm to 20 mm in length on the surface of the endodontic cone and in a layer which is between approximately 1 to 100 μm thick.

In another embodiment, the dental composition is used in a method of sealing a root canal, whereby the dental composition is mixed and then applied to the tooth root canal. In yet another embodiment, the dental composition is used in a method of making a pre-coated gutta percha point for use in a root canal procedure, whereby a gutta percha point is coated with a dental composition as described above.

### BRIEF DESCRIPTION OF THE DRAWINGS

For the purposes of illustrating the invention, there are shown in the drawings illustrative embodiments of various aspects of the invention. It is understood that these drawings depict only selected embodiments of the invention and are therefore not to be considered limiting of its scope.

FIG. 1 is a dental x-ray showing an endodontic cone within a root canal cavity, surrounded by the dental composition in accordance with one embodiment of the present invention.

FIG. 2. is a side view of a dual-chamber syringe in accordance with one embodiment of the present invention.

### DETAILED DESCRIPTION

While the specification concludes with the claims particularly pointing and distinctly claiming the invention, it is believed that the present invention will be better understood from the following description. All percentages and ratios used herein are by weight of the total composition and all measurements made are at 25° C. and normal pressure unless otherwise designated. All temperatures are in Degrees Celsius unless specified otherwise. The present invention can comprise (open ended) or consist essentially of the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention. Preferably, such additives will not be present at all or only in trace amounts. However, it may be possible to include up to about 10% by weight of materials that could materially alter the basic and novel characteristics of the invention as long as the utility of the compounds (as opposed to the degree of utility) is maintained. All ranges recited herein include the endpoints, including those that recite a range “between” two values. Terms such as “about,” “generally,” “substantially,” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value.

Prior to polymerization, the dental compositions of the present invention comprise a first component, a second component and at least one fumed silica. The first and second components, when polymerized, form an epoxy. The epoxy is a polymer; a term which includes polymers, copolymers, block copolymers and the like. The use of the term polymer is also often meant to include any portion of the monomers described herein after they have been mixed but before polymerization is completed as polymerization of the epoxy polymers of the invention are often spontaneous. Thus, as soon as mixing begins, polymerization begins and some portion of the mixture is already a polymer.

The first component is at least one type of polymerizable organic amide monomer. Without limitation, amide monomers may include a single amide group or multiple amide groups. The second component is at least one type of polymerizable organic diepoxide monomer. Without limitation, diepoxide monomers are composed a compound which includes two reactive epoxide groups. Polymerization occurs between alternating amide and diepoxide monomers. With the exception of the ends of a polymer chain, each amide monomer is bound to at least two epoxy groups, one each on two adjacent diepoxide monomers and each diepoxide monomer is bound two different amide monomers. It is also contemplated that the amide monomers and diepoxide monomers may be arranged in a random order, or arranged having a patterned order. This is particularly true where there are two or more different diepoxide monomers used and/or where there are two or more different amide monomers used.

Amides form epoxy polymers that may have desirable hardness and good impact strength. They may be chemically varied to obtain semi-flexible properties as well. Polyamides can be used as resin modifiers as well as curing agents. Many polyamides can cure at room temperature without blushing and show outstanding adhesion. Polymerizable organic amide monomers that may be useful in this invention include primary, secondary and tertiary amides, monoamides and polyamides. These include primary monoamides and secondary monoamides.

The polymerizable organic amide monomers are preferably present in an amount of about 5 to 70 percent by weight of the dental composition, more preferably in an amount of about 15 to 30 percent by weight of the dental composition, and most preferably in an amount of about 20 to 25 percent by weight of the dental composition. In a preferred embodiment, the amide monomers are resins comprising dimerized fatty acids and polyamines. Particularly useful are Versamid® 140, Versamid® 115 and Versamid® 125, manufactured by Cognis Corporation, located at 4900 East Avenue, Cincinnati, Ohio. Versamid® is a moderately-low viscosity, reactive amide monomer resin based on dimerized fatty acids and polyamines. It is typically used with solid or liquid epoxy resins to give tough, chemical resistant thermoset coatings using room temperature cure.

The amide monomers of the dental compositions of the present invention may also include, but are not limited to, one or more of the general formulas:

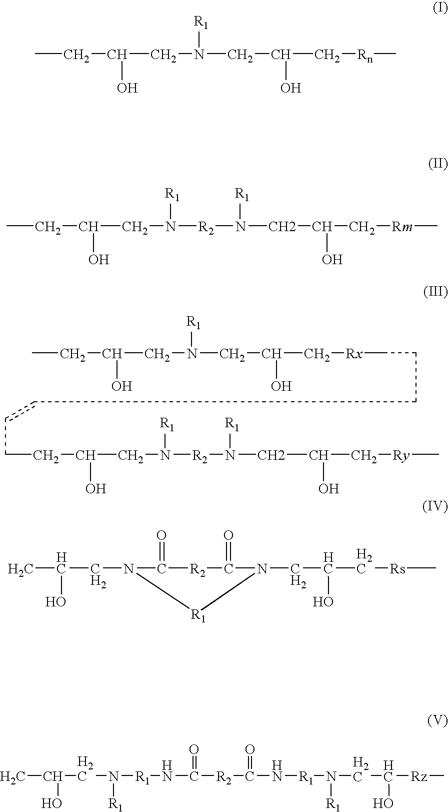

wherein R is a moiety formed from a diepoxide;

R₁ is a polymer segment formed by dimerization of fatty acid and polyamides, polymer segments containing amide groups, or a substituted alkyl, having from 2 to 20 carbon atoms, cycloalkyl, substituted cycloalkyl, aryl having from 2 to 20 carbon atoms, substituted aryl, arylalkyl, and substituted arylalkyl.

R₂ is a polymer segment formed by dimerized fatty acids and polyamides and/or polyamines, polymer segments containing amide groups, polymer segments containing amine groups, or a dysfunctional alkyl, substituted alkyl having from 2 to 18 carbon atoms, cycloakyl, substituted cycloalkyl, aryl having from 6 to 20 carbon atoms, substituted aryl, arylalkyl, and substituted arylalkyl, and n, m, x and y each independently is an integer from 1 to 1,000. When substituted, R₁ and R₂ are independently substituted with one or more alkoxy, halogen, nitrate, acyl or carboxy alkyl moieties.

The second component of the present invention is at least one polymerizable organic diepoxide monomer. Diepoxide monomers useful in dental filling compositions in accordance with the invention include diglycidyl ether of bisphenol-A (2,2-Bis[4-(2,3-epoxypropoxy)phenyl]propane), diglycidyl ether of bisphenol-F (an isomeric mixture of Bis[4-(2,3-epoxypropoxy) phenyl]methane and the 2,4-homologous (CIBA-Geigy)), butanediol diglycidyl ether, N,N-diglycidylaniline, and Δ³-tetrahydrophthalic acid (sometimes referred to as bis(2,3-epoxypropoxy) cyclohex-3-ene dicarboxylic ester).

Polymerizable organic diepoxide monomers are present in an amount of about 5 to 70 percent by weight of the dental composition, more preferably in an amount of about 15 to 30 percent by weight of the dental composition, and most preferably in an amount of about 20 to 25 percent by weight of the dental composition.

Fumed silica is used in connection with the first and second components as a filler. It may be added to either monomer component or packaged and added individually. Fumed silica helps to prevent separation between the first and second components of the dental composition of the present invention and any other fillers, if added. Fumed silica may also allow for a comparative reduction of silica content in the dental composition. Keeping the silica content low may improve the ability to later drill out a portion of the cured material if desired, such as to later place a post in a root canal during a crown restoration.

Fumed silica can be hydrophilic or hydrophobic. In the present invention, hydrophobic silica is preferred. Most preferred is Aerosil® R-972 manufactured by Degussa AG, located at Bennigsenplatz 1, 40474 Düsseldorf, Germany. As discussed on Degussa AG's website, www.degussa.com, Aerosil® R-972 is manufactured by a continuous flame hydrolysis process of silicon tetrachloride SiCl₄. During this process, SiCl₄ is converted into the gas phase and then reacts spontaneously and quantitatively in an oxyhydrogen flame with the intermediately formed water to produce the desired silicon dioxide. Aerosil's® are known for use in thickening polar liquids including epoxy resins, reinforcing silicone elastomers, enhancing loading levels, providing water-repelling properties, improving corrosion protection, lowering moisture adsorption, and improving dipersability.

In the case of Aerosil® R-972, freshly-produced hydrophilic Aerosil® 130 is converted with dimethyldichlorosilane (DDS) in a fluid-bed reactor. The silane reacts with the silanol groups primarily with the formation of Si—O—Si (CH₃)₂ units, and as a result the material acquires a hydrophobic character. The number of silanol groups is reduced during the treatment to about 30 percent of the initial value. Analogous reactions can also be carried out with other silanes and other hydrophilic grades.

In the present invention, Aerosil® R-972 or other fumed silica is preferably present in an amount of about 1 to 10 percent by weight of the dental composition, more preferably about 1 to about 5 percent by weight, and even more preferably in an amount of about 2.5 to 4.3 percent by weight, and most preferably in an amount of about 2.8 to 3.4 percent by weight of the dental composition. In each instance, the balance may be made up of monomers and other fillers, before mixing, or epoxy polymer following mixing. Therefore, the relative amount of epoxy polymer and other fillers in this dental material may range from as little as about 90 percent to as much as 100 percent. The relative amount of monomers and other fillers should add-up to about 90 to about 99 percent, although, the relative proportion of each monomer may vary as describe herein.

The first component—the polymerizable organic amide monomers, and the second component—the polymerizable organic diepoxide monomers, are preferably provided in a fixed ratio ranging from 1:3 by volume, amide monomers to diepoxide monomers, to 3:1, amide monomers to diepoxide monomers, ratio. And while a substantially 1:1 volume ratio of the first component to the second component is preferred, other ratios are contemplated by this invention.

Because the first component and the second component may react spontaneously to form an epoxy polymer, there is often a need to keep them separated before mixing. It is contemplated that they can be housed in separate individual packages or in separate chambers or portions of a single or multi-chamber package until such time as they are mixed to form the epoxy compositions of the present invention.

Besides the monomers and fumed silica as described herein, no other ingredients are needed to create the epoxy dental compositions of the present invention. That said, the dental compositions may also include other constituents including fillers, amines, colorants, antimicrobials, diluents, catalysts, modifiers, etc, keeping in mind that this composition is for use in a patient's mouth.

The use of fillers in the epoxy compositions can lower cost, reduce exotherms, extend shelf life, and achieve improvement in one or more of the epoxy resins properties, including improved machinability, improved abrasion resistance, improved impact strength, improved electrical properties, improved thermal conductivity, improved anti-settling, flow, or thixotropic properties. It should be understood that the use of fillers may result in the sacrifice of tensile, flexural and impact strength and this may limit the amounts used. Most fillers reduce the coefficient of thermal expansion and shrinkage in proportion to the amount of filler rather than the type of filler used.

In a preferred embodiment, fillers used in the present invention may also give the dental composition a high radiopacity (RO), allowing a dental device to be x-rayed. The radiopacity of the compositions of the present invention are preferably above 1 mm Al/mm dental composition, and more preferably above 3 mm Al/mm dental composition and most preferably above 5 mm Al/mm. At least one of the fillers used therefore preferably confers at least this level of radiopacity.

Fillers having Radiopacity useful in accordance with the invention, without limitation, include inorganic fillers such as Ag, TiO₂, La₂O₃, ZrO₂, BaSO₄, CaWO₄, BaWO₄, Fe₂O₃ and Bi₂O₃, lanthanide salts and polymer granulates. Reduced radiopaque fillers are also contemplated. By the term “reduced radiopaque filler(s)” it is meant to include fillers that do not provide substantial radiopacity, and in a preferred embodiment do not provide radiopacity above 2 mm Al/mm dental composition. Reduced radiopaque fillers may include, but are not limited to ZrO₂, SiO₂, ZrSiO, etc. In addition, the surface of the filler may be treated with substances such as epoxies, amines, etc. Generally, particle sizes used for such fillers are the same as is used conventionally in this art. Relatively fine particle sized fillers are preferred as they are easier to incorporate and have less tendency to settle. In a preferred embodiment, the average particle size of a filler material is preferably about 10 nm to about 10 μm. Fillers are preferably present in an amount of about 10 to 85 percent by weight of the dental composition, more preferably in an amount of about 20 to 75 percent by weight of the dental composition, and most preferably in an amount of about 40 to 55 percent by weight of the dental composition.

Amine monomers may also be used in place of, or in conjunction with the amide monomers and diepoxides to form an epoxy polymer. Without limitation, amine monomers may include a single amine group or multiple amine groups. Polymerizable organic amine monomers that may be useful in this invention include primary, secondary and tertiary amines, monoamines and polyamines. These include primary monoamines and secondary monoamines. As contemplated by this embodiment, the resulting epoxide polymer comprises at least one amide monomer, at least one amine monomer, a plurality of diepoxide monomers, and at least one fumed silica.

In one preferred embodiment, it is preferred that the first component—a mixture of the polymerizable organic amide monomers and the polymerizable organic amine monomers, and the second component—the polymerizable organic diepoxide monomers, are preferably provided in a fixed ratio ranging from 1:3 by volume, the first component to the second component, to 3:1, the first component to the second component, ratio. And while a substantially 1:1 volume ratio of the first component to the second component is preferred, other ratios are contemplated by this invention. The amine monomers may be present in an amount of about 1 to 25 percent by weight of the dental composition, and more preferably in an amount of about 5 to 15 percent by weight of the dental composition.

One further embodiment of the invention is a dental composition, as described herein, comprising an epoxy polymer comprising a mixture or reaction product of a mixture of diepoxide oligomers and a plurality of bridged dipolyamine monomers. A “diepoxide oligomer” is defined herein as any monomer or any oligomer of 2-5 monomer units (n=1-4 in the figure below), suitable for implantation in the mouth and containing two reactive epoxide groups. The term “oligomer” is used herein, even though it encompasses monomers in this context, to avoid confusion with the term monomer as used elsewhere.

Possible diepoxide oligomers include 1,6-hexanediol diglycidyl ether, 1,4-butanediol diglycidyl ether, 1,2-diglycidyl phthalate, or diglycidyl resorcinol ether, reacted with epoxide containing groups.

However, it is preferred that the diepoxide oligomers used to produce the ultimate epoxy polymer are a mixture of diepoxide oligomers having different lengths and/or compositions and are comprised primarily of bisphenol groups or units and two epoxide containing groups. The mixture of bisphenol containing diepoxide oligomers is preferably comprised of a combination of oligomers composed of single bisphenol groups and longer chains (as many as five bisphenol groups) both of which include, usually at the terminal ends, two reactive epoxy groups. These bisphenol containing diepoxide oligomers can be created by the following sample reactions using Bisphenol A and Bisphenol F as examples:

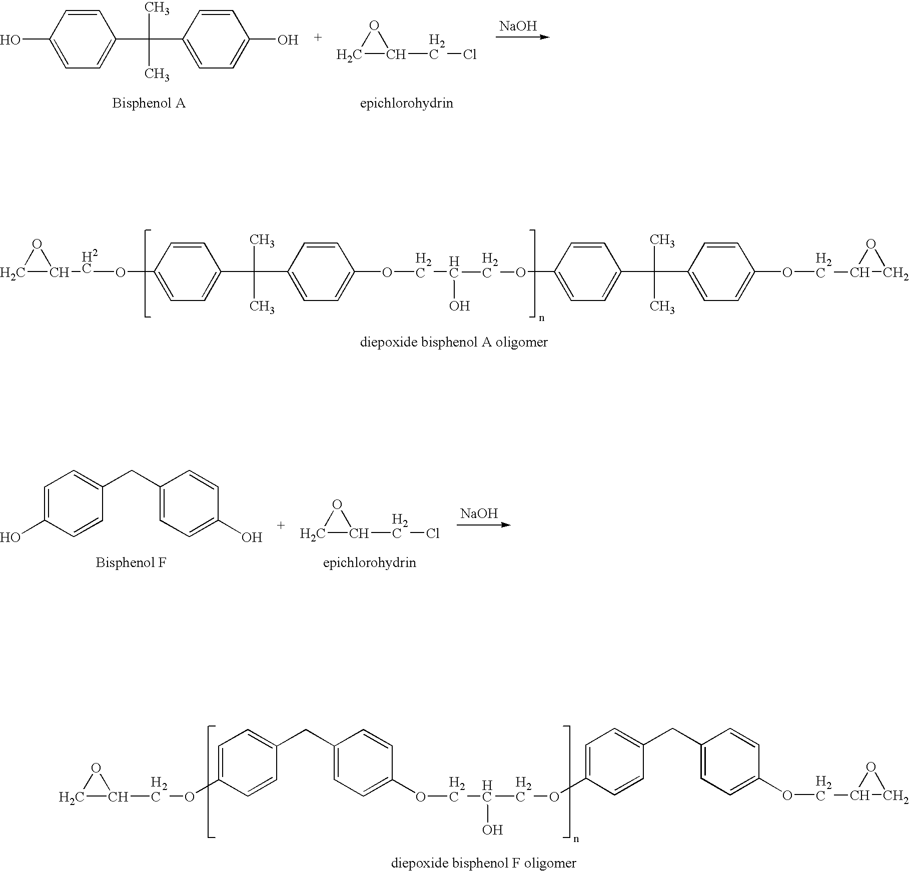

With respect to the above structures, n refers to a positive integer, preferably 0, 1, 2, 3, or 4. However, in another context, one which recognizes the variability of the distribution of various chain lengths in these oligomers, n refers to the average number of repeating units in the diepoxide oligomer. In this second context, n can be any value of 0 or greater and less than one, preferably between 0 and 0.5, more preferably between 0 and 0.25 and most preferably between 0.05 and 0.2. The average n number of a given diepoxide oligomer can be calculated from the epoxide equivalent weight (EEW) reported by the manufacturer. The EEW is the molecular weight per epoxy group. For example, Bisphenol A diglycidyl ether with n=0 has an EEW of 170 g/mol. Bisphenol A diglycidyl ether with n=1 has an EEW of 312 g/mol. By interpolating between these points, n values for most commercial products can be obtained.

While it is preferred that the bisphenol containing diepoxide oligomers contain between 1 and 5 bisphenol groups or units (used synonymously), it should be understood that the reactions creating the diepoxide oligomers are organic reactions and thus the diepoxide oligomers could include longer or shorter chains of bisphenol groups than those disclosed (i.e., n=2 does not mean that that is the only type of bisphenol diepoxide oligomer present). It should also be understood that the oligomers need not be, and in accordance with the invention, preferably are not, homogenous. It may be made of different members of a single bisphenol family or from different families. Thus, the mixture of bisphenol containing diepoxide oligomers could be, for example purposes only, a mixture of diepoxide bisphenol A oligomers, or a mixture of diepoxide bisphenol F oligomers, or a mixture of diepoxide bisphenol A oligomers and diepoxide bisphenol F oligomers, or a mixture of oligomers including subunits of both bisphenol A and bisphenol F, or a combination of any of the above mixtures.

Particularly well suited for use in the present invention are the Bisphenol A or Bisphenol F families, which include as examples:

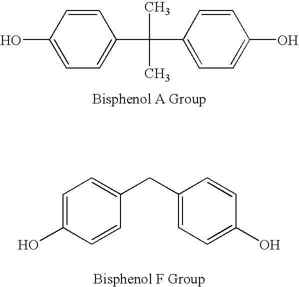

Assuming homogeneous oligomers, the number of Bisphenol A groups in each Bisphenol A diepoxide oligomer can range from 1 to 5. It is preferred that the mixture of Bisphenol A diepoxide oligomers contains primarily Bisphenol A diepoxide oligomers having only one Bisphenol A group and a relatively small amount of Bisphenol A diepoxide oligomers having more than one Bisphenol A group. This mixture is best represented by referring to the average number of Bisphenol A units per Bisphenol A diepoxide oligomer; i.e. the n value. Preferably the mixture of Bisphenol A diepoxide oligomers has an n value of 0 to 0.5, more preferably 0.1 to 0.25 and most preferably 0.14 to 0.20. It should be understood that even though the mixture is primarily Bisphenol A diepoxide oligomers having only one Bisphenol A group, variations in the relatively small amount of Bisphenol A diepoxide oligomers having more than one Bisphenol A group can have a great impact on the properties of said mixture.

Again assuming homogeneous oligomers, the number of Bisphenol F groups in each Bisphenol F diepoxide oligomer can range from 1 to 5. It is preferred that the mixture of Bisphenol F diepoxide oligomers contains primarily Bisphenol F diepoxide oligomers with only one Bisphenol F group and a relatively small amount of Bisphenol F diepoxide oligomers with more than one Bisphenol F group. This mixture is best represented by referring to the average number of Bisphenol F units per Bisphenol F diepoxide oligomer; i.e. the n value. Preferably the mixture of Bisphenol F diepoxide oligomers has an n value of 0 to 0.5, more preferably 0.05 to 0.25 and most preferably 0.07 to 0.13. It should be understood that even though the mixture is primarily Bisphenol F diepoxide oligomers having only one Bisphenol F group, variations in the relatively small amount of Bisphenol F diepoxide oligomers having more than one Bisphenol F group can have a great impact on the properties of said mixture.

It is preferred that a combination of bisphenol families be used to make up the diepoxide oligomers. Such combinations can have the same or different values of average number of bisphenol units per diepoxide oligomer. For example purposes only, the mixture of diepoxide oligomers could include combinations of oligomers containing a relatively low n value of Bisphenol A and a relatively high n value of Bisphenol F or a relatively high n value of Bisphenol A and a relatively low n value of Bisphenol F. It is particularly preferred that EPON 830, available from EV Roberts, is used to supply the Bisphenol A diepoxide oligomers and EPON 862, available from EV Roberts, is used to supply the Bisphenol F diepoxide oligomers.

When two different materials or families of materials are used, the relative amounts of Bisphenol A to Bisphenol F, for example, can vary from between 2:1 to 20:1 by weight. Preferably, the relative amounts of the first bisphenol to the second bisphenol are from between 3:1 to 10:1 and most preferably 7.4:1 by weight.

Without being bound by any particular theory, it is believed that the differing numbers of bisphenol monomers in the bisphenol oligomers, and/or a combination of different bisphenol families making up the bisphenol oligomers, retards the crystallization of the diepoxide oligomers. Use of differing numbers of bisphenol monomers to make up the bisphenol diepoxide oligomers has also been found to provide an acceptable viscosity for the plurality of diepoxide oligomers. Preferably the viscosity for the raw epoxy resin, before incorporation of fillers or reacting with the bridged dipolyamines, is between 200 and 2 Pa·s at 25° C., more preferably between 50 and 5 Pa·s at 25° C., and most preferably between 20 and 10 Pa·s at 25° C.

The mixed diepoxide oligomers are used as monomers and are reacted with bridged dipolyamine monomers to form the epoxy polymers of the invention. A bridged dipolyamine monomer, as used to produce the epoxy polymers of the invention, is defined as any monomer having at least two polyamino groups and a bridging group linking the polyamino groups.

Preferably the bridged dipolyamine monomers include two polyamine regions separated by a relatively long chain generally hydrocarbon containing region. Each polyamine region is comprised of at least two nitrogen atoms, preferably between three and five nitrogen atoms, often separated from each other by at least one carbon atom. When the polyamine containing region is linear or branched, it preferably contains primary and secondary amines. Preferable polyamine groups of this type include diethylene triamine, triethylene tetramine, and tetraethylene pentamine. However, preferred bridged dipolyamines include at least one cyclized region, generally an imidazoline group. Illustrative examples of the bridged dipolyamines are shown below:

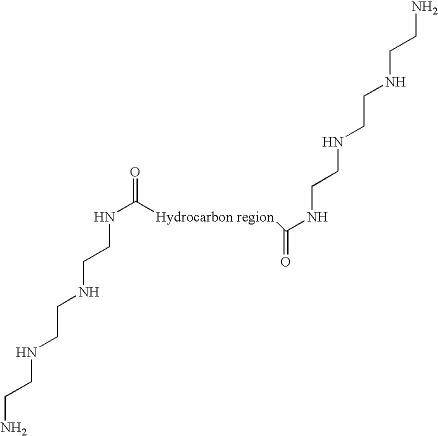

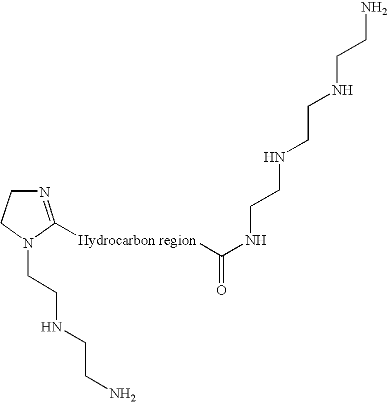

The bridged dipolyamines may also include both generally linear (which includes branched dipolyamines in this context) as well as generally cyclic groups in a single molecule (shown above on the right) and/or a generally linear bridged dipolyamine may be mixed with bridged dipolyamines including at least one cyclized group. In the above figures, for example, both structures may be present. Indeed, there are believed to be advantages to the use of such combinations where the amount of bridged polyamines containing a cyclized group is at least about 20% by weight, more preferably at least about 50% by weight, even more preferably at least about 60% by weight and most preferably 70% by weight or more. It is noted that, when reacted to form an epoxy polymer as explained herein, some or all of the cyclized groups of the bridged polyamines may de-cyclize. Therefore, to meet the aforementioned amounts of cyclized groups, one need not find the stated amounts of cyclization in the cured product, provided such amounts were present in the bridged polyamine starting material. Thus, if an epoxy polymer is described as including a cyclized group, for example, if it were described as comprising “at least about 20% by weight of a plurality of bridged dipolyamine monomers”, that description is meant to encompass a cured epoxy polymer even where none of the bridged dipolyamines retain this cyclized form, but where the bridged dipolyamine monomers used to make the polymer included at least about 20% by weight of said plurality of bridged dipolyamine monomers mixed with up to 80% by weight of bridged dipolyamines, prior to polymerization.

The polyamine regions are bridged or connected by a hydrocarbon region having at least 28 or more carbon atoms and preferably at least 32 or more carbon atoms; most preferably 36 carbon atoms in the region. This region need not be completely hydrocarbon in nature as it can be substituted or unsubstituted, and may contain ester and or ether linkages or groups capable of forming same. See, for example, the carbonyl groups in the figure above. These groups may be used to link the hydrocarbon region to the polyamine regions and/or may be used to allow smaller groups to be linked together to produce a longer hydrocarbon region. Additionally, the hydrocarbon region can be saturated or unsaturated, straightened or branched and, as in one preferred embodiment, can be derived from a dimer of fatty acids or alcohols. Preferred hydrocarbon regions include glutaric acid, azelaic acid, or dimerized fatty acids. Most preferably, the bridged dipolyamine monomer used is Versamid 140, available from EV Roberts. Without being bound by any particular theory, it is believed that, when polymerized with the plurality of diepoxide blocks, the polyamines permit cross-linking in three dimensions such that a matrix is formed as opposed to a strictly linear molecule, and the long chain of the carbon region provides more variability in the cross-linking, allowing one bridged dipolyamine to be linked to two or more diepoxide oligomers which are relatively far apart. Without being bound by any particular theory, it is believed that the cross linking of the epoxy polymer increases the hardness of the material.

The following figures illustrate some of the various ways the diepoxide oligomers and bridged dipolyamine monomers can react to form the epoxy polymer of the instant invention:

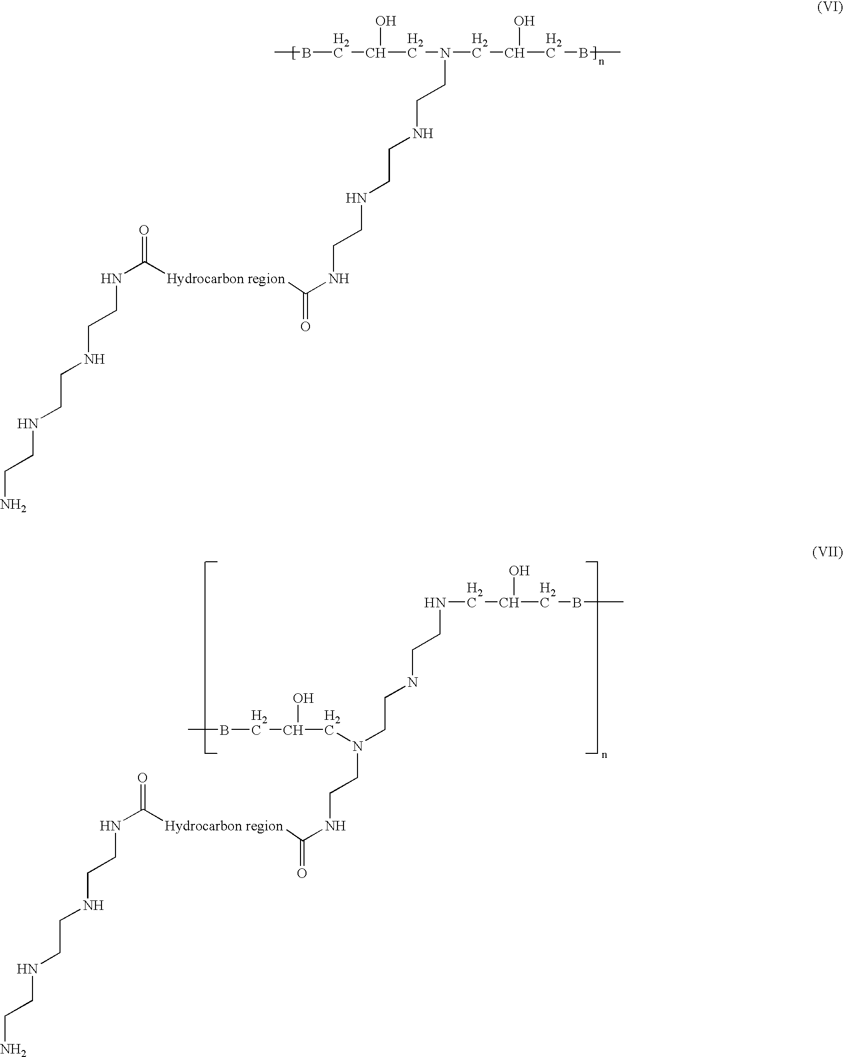

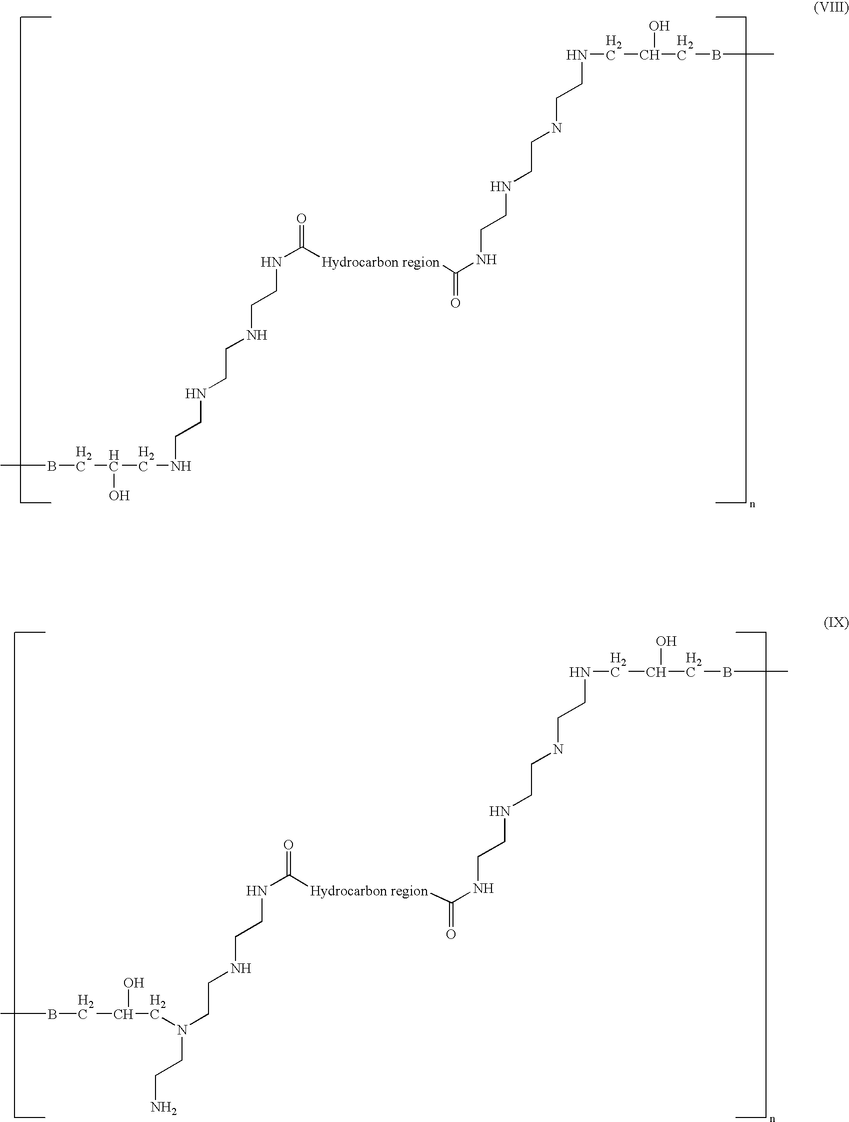

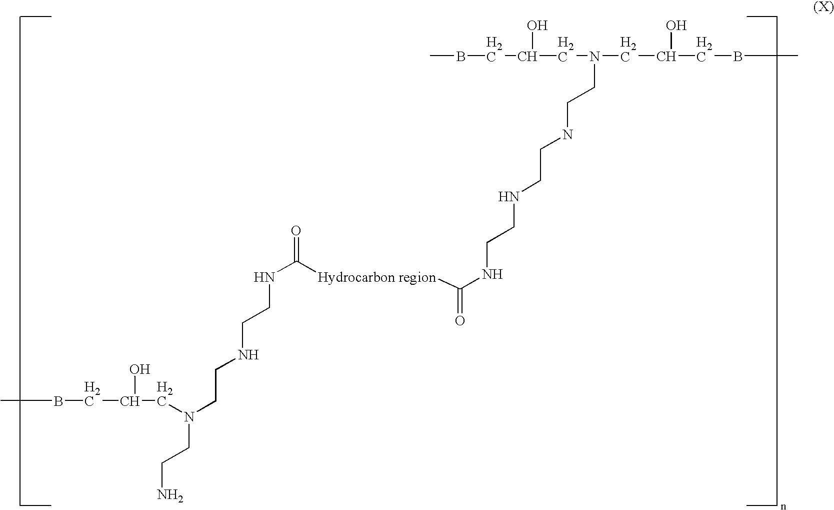

where B represents the remainder of the diepoxide oligomer (which includes the oligomer and the remaining epoxide group either unbound or bound to an additional bridged dipolyamine) and n represents the fact that each figure is simply a piece of an epoxy polymer chain, and that the epoxy polymer continues beyond the brackets. Thus, the above figures illustrate that the diepoxide oligomers can bind to any amine on either, or both, polyamine region of the bridged dipolyamine and that a single bridged dipolyamine can bind to multiple diepoxide oligomers. It should be noted, that the above figures are illustrative only, and that the polymer chains can continue with either the same or different units as those shown in the figures.

The amount of diepoxide oligomers to bridged dipolyamine monomers used can be the same as those described herein for the amide monomers and epoxy monomers. However, preferably they range from about 3:1 to about 1:3, more preferably about 1.5:1 to about 1:1.5, and most preferably 1:1 diepoxide oligomers to bridged dipolyamine monomers. Additionally, the fillers and other materials described herein, can be used in the same way, and in the same amounts, as disclosed for other epoxy polymers described herein.

As opposed to prior art compounds, the epoxy polymers of this aspect of the instant invention require no initiator to promote binding between the diepoxide blocks and the bridged dipolyamine monomers. Without being bound by any particular theory, it is believed that the crosslinking of the epoxy polymers of the instant invention also increase the hardness of the epoxy polymer. Additionally, where the bridged dipolyamine monomers, and hence the ultimate epoxy polymer formed therefrom, contain imidazoline groups, over time and when exposed to moisture, the ring structures have been found to de-cyclize. Without being bound to any particular theory, it is believed that when the epoxy polymer is implanted in the root canal, the cyclized groups in the epoxy polymer react with water, decyclizing, which inhibits shrinkage of the epoxy polymer and indeed causes the epoxy polymer to expand slightly in that area. Without being bound by any particular theory, it is also believed that that the use of cyclized groups also slows the curing of the epoxy polymer thereby allowing more working time with the epoxy polymer before it sets.

This aspect of the instant invention can be used with any other optional ingredients discussed herein and in any dental practice discussed herein such as, for example, the affixing of endontic cones in teeth.

A colorant is any substance that imparts color to another material or mixture. Colorants may be added for aesthetic purposes, making the resulting product a natural or preferred color. Colorants may include dyes or pigments. Most pigments are insoluble, inorganic powders, the coloring effect being a result of their dispersion in a solid or liquid medium. Most dyes are soluble synthetic organic products which are chemically bound to and actually become a part of the applied material. The use of either dyes or pigments, or both are contemplated. Preferred colorants useful in the present invention include titanium dioxide, iron oxide or other insoluble organic or inorganic colorants. These colorants are present preferably in amount about 0.01 to 5 percent by weight of the dental composition, and more preferably from about 0.01 to 1 percent by weight of the dental composition.

The dental composition of the present invention may optionally include one or more antimicrobial agents to assist in cleansing and sterilizing the root canal and to prevent later infection. Examples of suitable antibacterial agents include organohalogens, antibiotics, alkali metal hydroxides, alkaline earth metal oxides, and alkaline earth metal hydroxides. Examples of antibacterial organohalogens include 1,1′-hexamethylene bis(5(p-chlorophenyl)biguanide), cetyl pyridinium chloride, benzalkonium chloride, and cetyl pyridinium bromide. Examples of suitable antibiotics include: 4′sulfamoylsulfanilanilide, 3-amino-6-(2-(5-nitro-2-furyl)vinyl)pyridazine, trans-pseudomonic acid, xanthomycin, alpha-amino-p-toluene sulfonamide, alpha-azido benzyl penicillin, penicillin O, penicillin N, monopropionyl erthromycin, and erythromycin 9(O-((2-methoxy ethoxy)methyl)oxime. Examples of suitable alkali metal hydroxides include sodium hydroxide and lithium hydroxide. Examples of suitable alkaline earth metal oxides include calcium oxide, magnesium oxide, barium oxide, and strontium oxide. Examples of suitable alkaline earth metal hydroxides include calcium hydroxide, magnesium hydroxide, barium hydroxide, and strontium hydroxide. One preferred antimicrobial agent is silver, as it kills microorganisms and is compatible with dental tissue. Another preferred antimicrobial agent is calcium hydroxide. Other preferred antimicrobial agents include triclosan and chlorhexidine salts. The antimicrobial agent may be included in a preferred amount of about 0.01 to about 3 percent by weight of the dental composition, more preferably in an amount of about 0.05 to about 1.2 percent by weight of the dental composition, and most preferably in an amount of about 0.1 to about 1 percent by weight of the dental composition.

The dental composition may also include one or more reactive or non-reactive diluents. A diluent is used primarily to reduce viscosity. Adding a diluent also permits higher filler loading and gives better wetting and impregnation. Preferably, the diluents should contribute substantial viscosity reduction at low concentrations, and be non-reactive with the dental composition under normal storage conditions. Diluents contemplated for use in the present invention may include, but are not limited to, butyl glycidyl ether, cresyl glycidyl ether, 2-thylhexyl glycidyl ether, etc. The diluents may be included in a preferable amount in a range from about 0.1 to 5 percent by weight of the dental composition.

The dental composition may also include inert organic or inorganic liquids or gels, including oils. However, the addition of such liquids or gels, and in particular, silicone oils and mineral oils, can reduce adhesion, especially to root canal tissues, and reduce shelf life of the product due to separation of the oil over time. Thus, it is preferred that the dental composition contain as little inert liquids or gels, and especially silicone oils and mineral oils, as possible. In a preferred embodiment, substantially no inert organic or inorganic liquids or gels are used; preferably less than about 1.0 percent, more preferably less than 0.01 percent by weight of the dental composition.

Catalysts may also be added in amounts preferably about 0.01 to 5 percent by weight of the dental composition or dental polymer, to initiate or speed up polymerization. Catalysts may also be included to speed up reaction time of the components. While it is preferred that the components of the dental compositions of the present invention substantially react within one to six hours from their mixing, and more preferably within two to four hours from their mixing.

It is also within the scope of the invention to include other additives or adjuvents as desired in order to impart a desired property, such as less or non-radio opaque fillers, diluents, dyes or plasticizers.

The dental composition of the present invention may be used alone as a sealant to fill the cavity of a root canal, and can be used for either permanent or temporary use. The substantially polymerized dental polymer may be extracted from a root tooth canal with the use of an organic solvent, including CHCl₃ and C₂H₅OH. The amount of product used is preferably enough to fill the root cavity without substantial overflow, taking into account expansion during dissolution.

The dental composition may also function as a coating onto an endodontic cone. Endodontic cones are cylindrical-shaped plugs that fit into a root canal opening. Once in place, a dental resin composition is used to bond the endodontic cone to the root canal tissue, thus filling the cavity of the root canal. Endodontic cones are most widely made of gutta percha, but may be made of other materials. Gutta percha is a natural latex obtained from Palaquium gutta and several other evergreen trees of East Asia. The latex, collected by felling or girdling the tree, is allowed to coagulate and is then washed, purified, and molded. Gutta-percha is a polyterpene, i.e., a polymer of isoprene, but not very elastic. Gutta-percha is an excellent nonconductor and is often employed in insulating marine and underground cables, golf-ball coverings, surgical appliances, and adhesives. Gutta-percha is softened by hot water (70° C.), and when soft it may be easily cut or molded into various shapes making it ideal to be uniquely bent to the shape of an individual's root canal.

While endodontic cones have been used in root canal procedures for quite some time, it is often difficult to bond the endodontic cone to the root canal tissue due to their differences in hydrophobic and hydrophilic properties. The epoxy compositions of the present invention address these problems by providing superior bonding. The epoxy formulations of the present invention can be applied to the cone by inserting the uncoated cone into a root canal filled with the dental compositions of the present invention. In addition, or in the alternative, the cone can be pre-coated with the mixed, and thus polymerizing, dental composition material to form a coated cone which is then inserted into the root canal where the monomers on the cone and those in the canal are allowed to cure.

In yet another embodiment, the cone can be pre-coated and the coating allowed to cure, partially or totally, prior to being used. This dried pre-coating can improve adhesion when using the compositions of the present invention, and even when the thus coated cone is used in combination with some types of dental adhesive other than those described herein. Thus, by pre-coating the endodontic cone with the dental compositions of the present invention, a better bond results between it, and the dental compositions. It is contemplated that the pre-coated endodontic cone of this invention is used with either the dental compositions of the present invention as a sealant to the root canal, or some other sealant.

The endodontic cone may be coated with unfilled dental composition, containing no fillers or other additives besides the amide monomers and diepoxide monomers, or filled dental composition, as previously discussed, containing one or more fillers or other additives. In one embodiment wherein no fillers are added, it is preferred that the first component, polymerizable organic amide monomers are preferably present in an amount of about 20 to 80 percent by weight of the dental polymer, and more preferably in an amount of about 30 to 70 percent by weight of the dental polymer. In this embodiment, it is preferably that the second component, polymerizable organic diepoxide monomers are present in an amount of about 20 to 80 percent by weight of the dental polymer, and more preferably in an amount of about 25 to 70 percent by weight of the dental polymer.

In another embodiment, wherein filler is added to either the first component or second component separately, or together, it is preferred that the inorganic filler is present in an amount of about 5 to 70 percent by weight of the dental polymer, and more preferably in an amount of about 10 to 50 percent by weight of the dental polymer.

Coating may be attained through a spray process, brushing, dipping the endodontic cone into the dental composition, or any other coating technique, and then allowing the dental composition to polymerize. The endodontic cone may be coated, allowed to polymerize, and stored for a time before a root canal procedure, coated and allowed to polymerize during a root canal procedure, or simply coated, and applied, allowing polymerization within a patient's mouth.

Before application of the dental composition, or after curing is complete, the surface of the endodontic cone, or the surface of the coating may be texturized (e.g., a lattice framework, or dimpling) to increase the surface area and provide greater retention and bonding of the dental composition coating and/or the coating and the adhesive material within the root canal.

Preferably, the dental adhesive composition is well distributed over at least a portion of one of the surfaces of the cone, and more preferably an area of about 0.1 mm to 40 mm in length from the pointed tip along the surface of the endodontic cone, and even more preferably over an area of about 0.1 mm to 20 mm in length from the pointed tip along the surface of the endodontic cone. The dental composition is preferably well distributed on the surface of the endodontic cone approximately 1 to 100 μm thick, and more preferably 2-40 μm thick. It is understood however that when the coating is applied manually, it may be thicker than desired and may be applied less evenly over the surface.

The dental compositions of the present invention may also be used as dental sealers in connection with the use of an endodontic cone, thus surrounding the endodontic cone and bonding the cone to the root canal tissue. In this application, it is preferred that the dental composition only comprise the first and second components, the monomers of the dental compositions in accordance with the present invention. However, the addition of fumed silica and other fillers are also contemplated.

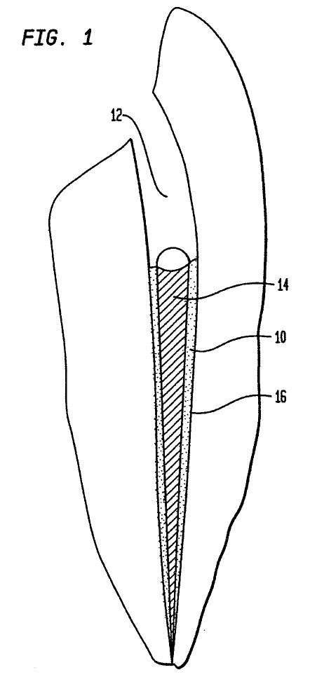

As shown in FIG. 1, the dental composition (10) may be placed within the root canal cavity (12) surrounding and bonding the gutta percha point (14) to the root canal tissue (16). The amount of dental composition used in connection with an endodontic cone to fill the cavity of a root canal will be dependent upon the size of the root canal and the endodontic cone.

The first and second components, the monomers of the dental compositions in accordance with the present invention, must be kept separate before mixing, as mixing induces spontaneous polymerization. Any other ingredients, including the fumed silica, may be kept separately from either or both the first and second components, combined with either of the first or second separately housed components, or combined with other ingredients housed separately from the first and second components. Thus, for example, the fumed silica could be mixed with the amide monomers, or the diepoxide monomers. It could be stored with neither; being housed in a completely separate container, or it could be distributed in both. In an additional and again non-limiting example, the fumed silica could be mixed with the amide monomer and the filler and a colorant mixed with the diepoxide. When the monomers and their included material are measured and mixed in the desired proportions, the resulting formulation contains a desired amount of each component. All of the fillers and other additives or excipients could be mixed with, for example, the diepoxide monomers and the amide monomers stored separately. All of the excipients and fumed silica could be stored in one or more separate containers with the monomers being stored separately from each other and separately from the fumed silica and the excipients.

The first and second components must be housed separately because spontaneous polymerization takes place from the point of contact between the first component and the second component. The product is substantially polymerized when the components have fully reacted, to the extent that they can in a reasonable period of time, resulting in a dental polymer in accordance with the present invention. It is preferred that substantial polymerization take place within one to six hours from the point of mixing the first and second components. The components in the dental composition undergo reaction until the point of substantial polymerization. When used as a dental sealant, it is during this reaction time that the material is inserted within the root canal. In this type of application, substantial polymerization takes place within the patient's mouth (except for any pre-polymerized pre-coating). This dental polymer is readily removable from the root canal by drilling or solubilizing in an organic solvent or diluent.

Also as previously noted, there are many options for the mixing and storage of the various components. Thus, it is contemplated that a user may place all of the ingredients together, one at a time, or in any combination thereof, on a mixing pad, in a mixing bowl, or other mixing apparatus, and then manually mix them using a spatula or other mixing tool to initiate polymerization prior to application in a patient's mouth.

Although manual mixing is contemplated, other mixing alternatives may also be used. A preferable option is the use of a multi-chamber package wherein most, if not all of the components are housed. This multi-chamber package may be in the form of a syringe. In a particularly preferred embodiment, all the components are stored in one or the other of two chambers of a dual-chamber package system, such as a dual-chamber syringe. Where each component is housed is not critical so long as the two monomers are separated. Where the other components will be stored may depend upon a number of factors including volume, viscosity, dispersability and the like.

The components may be removed and measured from any container by known techniques. If the container is bottle, the contents may be poured. If the container is a jar, the contents may be removed by a spoon. If the container is a squeeze tube, the contents may be expelled by squeezing the tube. In a preferred embodiment, a multi-chamber (at least a dual-chamber) syringe is used which has at least one common opening and at least one plunger for pushing the contents of the chambers out so that they can be mixed and used. Multiple plungers, at least one for each chamber, may be provided. And each chamber can have its own opening for the contents to be expelled through. In an addition, the syringe can be fitted with multiple plungers, each of which is interconnected so a user would depress a single surface, and that will move all of the plungers in each of the chambers. Depending upon the size of the chambers and plungers, the amount expelled from each chamber could be the same by weight or volume, or could be different.

The multi-chamber syringe may also include a mixing chamber into which the components housed within the various chambers are expelled into the chamber and allowed to mix before being expelled for use. The multi-chamber syringe may also include an auto-mixer housed within the mixing chamber, for mechanically mixing the components before being expelled from the syringe. Such auto-mixers may include products such as ML 2.5-08-D or ML 2.5-12-D (V02) with or without an intra-oral-root canal tip (such as IOR 209-20 (V01)). The multi-chamber device may also have measuring indicia, or a method for delivering a pre-desired amount of each monomer, together with any additives and fumed silica. The multi-chamber device may also be adjustable for mixing the components in a variety of ratios.

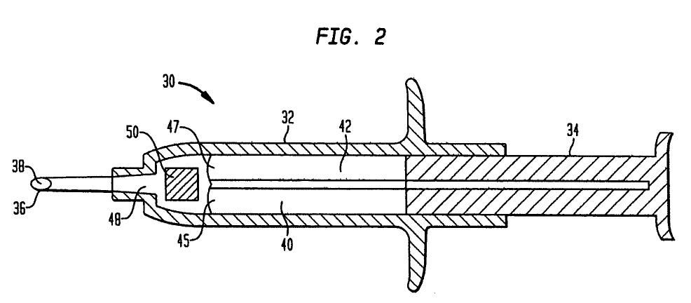

As shown in FIG. 2, one embodiment contemplated by the present invention includes a dual-chamber syringe (30) comprising a barrel (32), a plunger (34) and an extrusion point (36) having an opening (38) for expelling the dental composition of the invention. Within the barrel (32) is a first chamber (40) for housing the first component of the dental composition, a second chamber (42) for housing the second component of the dental composition. The base (44) of the first chamber (40) defines an orifice (45) and the base (46) of the second chamber (42) defines an orifice (47), through which the first component and second component may be deposited from the first chamber (40) and the second chamber (42), respectively. The barrel (32) also comprises a mixing chamber (48) wherein the first chamber (40) and the second chamber (42) release their respective components before mixing and expelling the mixture from the opening (38) in the extrusion point (36). The mixing chamber (48) may also include an auto-mixer (50) for mechanically mixing the components before expulsion. A double-barreled syringe, each barrel including a chamber for retaining each of the first and second components of the present invention is also contemplated.

In operation, a user depresses the plunger (34) of the syringe (30), housing the first and second components, thereby moving the plunger toward the opening (38). As the plunger moves within the dual chambers (40 and 42), the first component and the second component housed therein, are extruded through the respective orifices (45 and 47) located at the base (44) of the first chamber (40) and the base (46) of the second chamber (42), into the mixing chamber (48) within the barrel (32) of the syringe (30). Here, the components contact each other, initializing polymerization. They may be mixed in the mixing chamber (48) as they are extruded through the opening (38) of the syringe (30) due to force on the plunger (34). Alternatively, the components may be mixed via an automixer (50) located within the mixing chamber (48) of the barrel (32) of the syringe. It is also contemplated that the components may be extruded and then manually mixed.

The dental composition of the invention preferably has a viscosity between 2,500 and 100 poise at 25° C., more preferably between 2,000 and 500 poise at 25° C., and most preferably between 1,500 and 1,000 poise at 25° C., and is preferably passed through a 1 mm diameter canal of an intra-oral-root canal tip into a pulp chamber of a tooth.

In a most preferred embodiment, the dental composition of the present invention comprises an epoxy based root canal sealer that has an enhanced shelf life in comparison to other root canal sealer products. By enhanced shelf life it is meant that the composition is resistant to crystallization, meaning that the composition remains in liquefied form, over a storage period of at least one year, more preferably two years, and most preferably three years. In addition, it has been found that this combination of ingredients is also resistant to settling out. By “settling out” it is meant that the ingredients stay in solution or suspension and do not separate over a period of at least one year, and more preferably two years, and most preferably three years. In this most preferred embodiment, the dental composition comprises an epoxy polymer preferably comprising about 20 to 25 percent by weight of a plurality of polymerizable organic amide and/or amine monomers, and about 20 to 25 percent by weight of a plurality of polymerizable organic diepoxide monomers, about 2.8 to 3.4 percent by weight of at least one fumed silica, at least one antimicrobial agent, at least one inorganic filler, and at least one additional ingredient including organic fillers, colorants, diluents, catalysts, or modifiers. In addition to the foregoing, the most preferred embodiment contains substantially no silicone or mineral oil.

The following examples further illustrate the present invention. They are not intended to be limiting in any way. All of the following percentages refer to weight percent.

### EXAMPLE 1

The dental composition was prepared in a two-part form. Part one of the paste contained 26% Versamid® 140, 2% Aerosil® R-972, 71% bismuth trioxide, and 1% silver. Part two of the sealer paste contained 100% diglycidyl ether of bisphenol-A. The two pastes were packed to a double syringe 5 ml plastic syringe (Mixpac, Switzerland). The pastes were expressed out premixed through mixers matching the syringe (such as ML 2.5-08-D, ML-25-16-S(V01), ML 2.5-12-D (V02)) with an intra-oral-root canal tip (such as IOR 209-20 (V01)).

After mixing, the sealer paste set readily within 4 hours at a temperature of approximately 37° C. The radiopacity was 7.8 mm Al/mm dental composition.

### EXAMPLE 2

The dental composition was prepared in a two-part form. Part one of the paste contained 37% Versamid® 140, 2% Aerosil® R-972, and 61% bismuth trioxide. Part two of the sealer paste contained 38% diglycidyl ether of bisphenol-A, 1% Aerosil® R-972, 49% calcium tungstate, and 12% zirconium oxide. The two pastes were packed to a double syringe 5 ml plastic syringe (Mixpac, Switzerland). The pastes were expressed out premixed through mixers matching the syringe (such as ML 2.5-08-D, ML-25-16-S(V01), ML 2.5-12-D (V02)) with an intra-oral-root canal tip (such as IOR 209-20 (V01)).

The sealer paste set readily within 4 hours and the working time was about 2 hours at a temperature of approximately 37° C. The radiopacity was 8.3 mm Al/mm dental composition.

### EXAMPLE 3

The dental composition was prepared in a two-part form. Part one of the paste contained 35% Versamid® 140, 2% Aerosil® R-972, 63% bismuth trioxide. Part two of the sealer paste contained 38% diglycidyl ether of bisphenol-A, 1% Aerosil® R-972, and 61% bismuth trioxide.

The pastes were mixed on mixing pad using a spatula. They were mixed with a one to one volume ratio, and the radiopacity was 10.6 mm Al/mm dental composition.

### EXAMPLE 4

The dental composition was prepared in a two-part form. Part one of the paste contained 35.4% Versamid® 140, 2.6% Aerosil® R-972, and 62% bismuth trioxide. Part two of the sealer paste contained 37% diglycidyl ether of bisphenol-A, 2.6% Aerosil® R-972, 58.7% bismuth trioxide, and 1.5% titanium dioxide. The two pastes were packed into a double syringe 5 ml plastic syringe (Mixpac, Switzerland). The pastes were expressed out premixed through mixers matching the syringe (such as ML 2.5-08-D or ML 2.5-12-D (V02)) with an intra-oral-root canal tip (IOR 209-20 (V01)).

The sealer paste set readily within 4 hours and the working time was about 2 hours at a temperature of approximately 37° C. At ambient temperature around 25° C., the gel time was about 4 hours and the set time was about 8 hours. The radiopacity was 10.4 mm Al/mm dental composition.

### EXAMPLE 5

The dental composition was prepared in a two-part form. Part one of the paste contained 34.38% Versamid® 140, 3.6% Aerosil® R-972, and 62% bismuth trioxide, and 0.017% iron oxide. Part two of the sealer paste contained 32% diglycidyl ether of bisphenol-A, 3.8% Aerosil® R-972, and 64.2% bismuth trioxide. The two pastes were packed into a double syringe 5 ml plastic syringe (Mixpac, Switzerland). The pastes were expressed out premixed through mixers matching the syringe (such as ML 2.5-08-D or ML 2.5-12-D (V02)) with an intra-oral-root canal tip (IOR 209-20 (V01)).

The sealer paste set readily within 3 hours and the working time was about 1 hour at a temperature of approximately 37° C. At ambient temperature around 25° C., the gel time was about 3 hours and the set time was about 6 hours. The radiopacity was 11.3 mm Al/mm dental composition.

### EXAMPLE 6

The dental composition was prepared in a two-part form. Part one of the paste contained 43% Versamid® 140, 4.7% Aerosil® R-972, and 52.25% bismuth trioxide, and 0.05% iron oxide. Part two of the sealer paste contained 45.1% diglycidyl ether of bisphenol-A, 6.2% diglycidyl ether of bisphenol-F, 5.2% Aerosil® R-972, and 43.5% bismuth trioxide. The two pastes were packed into a double syringe 5 ml plastic syringe (Mixpac, Switzerland). The pastes were expressed out premixed through mixers matching the syringe (such as ML 2.5-08-D or ML 2.5-12-D (V02)) with an intra-oral-root canal tip (IOR 209-20 (V01)).

### EXAMPLE 7

The dental composition was prepared in a two-part form. Part one of the paste contained 44% Versamid® 140, 3.5% Aerosil® R-972, and 50.45% bismuth trioxide, 0.05% iron oxide, and 2% zirconium dioxide. Part two of the sealer paste contained 41.8% diglycidyl ether of bisphenol-A, 5.7% diglycidyl ether of bisphenol-F, 3.9% Aerosil® R-972, 38.6% bismuth trioxide, and 10% zirconium oxide. The two pastes were packed into a double syringe 5 ml plastic syringe (Mixpac, Switzerland). The pastes were hand mixed in ratios of 2:1, 1:1, and 1:2. All mixtures set properly.

### EXAMPLE 8

The dental composition was prepared in a two-part form. Part one of the paste contained 44% Versamid® 140, 3.5% Aerosil® R-972, and 50.45% bismuth trioxide, 0.05% iron oxide, and 2% zirconium dioxide. Part two of the sealer paste contained 41.8% diglycidyl ether of bisphenol-A, 5.7% diglycidyl ether of bisphenol-F, 3.9% Aerosil® R-972, 38.6% bismuth trioxide, 8% zirconium oxide, and 2% triclosan. The two pastes were mixed by hand in a 1:1 ratio.

### EXAMPLE 9

### Coating Gutta Percha Points

Commercially available gutta percha points from DIADENT were coated with the dental composition of the present invention, from 0 mm to 20 mm length. The volume ratio of bisphenol A-diglycidylether to Versamid® 140 of the cured coating was 1:1. SEM analysis revealed a film thickness of 20 μm.

### EXAMPLE 10

Commercially available gutta percha points from DIADENT were coated with the dental composition of the present invention, from 0 mm to 20 mm length. The volume ratio of bisphenol A-diglycidylether versus Versamid® 140 of the cured coating was 1:1. SEM analysis revealed a film thickness of about 2 μm.

An extracted tooth was then instrumented with the safesider technique and then fitted with the coated gutta percha point. The dental composition was applied with an EZ-Fill Bi-Spiral®. SEM analysis revealed excellent adaptation between the point, coating, and dental composition.

### EXAMPLE 11

ADA Testing in accordance with ADA Specification No. 57, was performed on an epoxy polymer material composed of a reaction mixture of equal parts, by volume, of the following Epoxy and Amine components:

| | Epoxy component (% by | Amine component (% by |
|---|---|---|
| Ingredients | weight) | weight) |
| Versamid | | 44.0% |
| 140 | | |
| EPON 830 | 41.4% | |
| EPON 862 | 05.6% | |
| Bi₂O₃ | 44.6% | 49.53% |
| ZrO₂ | 05.4% | 03.4% |
| Aerosil R- | 03.0% | 03.0% |
| 972 | | |
| Fe₂O₃ | | 00.07% |

The working time, setting time, flow, film thickness, dimensional stability, solubility and radiopacity of the aforementioned epoxy polymer was measured in accordance with ADA Specification No. 57. Two samples were tested for working time and three samples were tested for the remaining tests. The working time was greater than 30 minutes for the 36×37 mm sample and greater than 40 minutes for the 36×36 mm sample. The other test results are as follows:

| Sample | |
|---|---|
| | Setting Time |
| 1 | 2 hr 45 min |
| 2 | 2 hr 15 min |
| 3 | 2 hr 30 min |
| Average | 2 hr 30 min |
| | Flow (mm) |
| 1 | 36 × 37 |
| 2 | 39 × 40 |
| 3 | 38 × 37 |
| Average | 38 |
| | Film Thickness (μm) |
| 1 | 40 |
| 2 | 36 |
| 3 | 32 |
| Average | 36 |
| | Dimensional Stability (% expansion) |
| 1 | 0.09 |
| 2 | 0.07 |
| 3 | 0.10 |
| Average | 0.09 |
| | Solubility (%) |
| 1 | −0.54 |
| 2 | 0.99 |
| 3 | 0.46 |
| Average | 0.30 |
| | Radiopacity (mm Al) |
| 1 | 8.0 |
| 2 | 7.5 |
| 3 | 8.0 |
| Average | 7.8 |

The afore-discussed test results meet or exceed ADA specifications for Endontic Sealing Materials.

### EXAMPLE 12

The epoxy polymer material from Example 11, formed as the reaction product of the following Epoxy and Amine components:

| | Epoxy component (% by | Amine component (% by |
|---|---|---|
| Ingredients | weight) | weight) |
| Versamid | | 44.0% |
| 140 | | |
| EPON 830 | 41.4% | |
| EPON 862 | 05.6% | |
| Bi₂O₃ | 44.6% | 49.53% |
| ZrO₂ | 05.4% | 03.4% |
| Aerosil R- | 03.0% | 03.0% |
| 972 | | |
| Fe₂O₃ | | 00.07% |

was subjected to strength testing. AH-Plus, obtained from Dentsply, was also tested for comparative purposes. Three diameter inch samples of each the two materials were measured using a method according to ADA Specification 27. The samples were formed in Teflon molds and allowed to cure for 24 hours at 37° C. A force was applied to each sample with a universal testing machine (810 MTS), at a crosshead speed of 0.635 cm/minute until the samples were crushed. The results of this test are as follows:

| | Average | Average |
|---|---|---|
| | Diameteric | Compressive |
| | Tensile | Strength |
| | Strength (MPa) | (MPa) |
| Example 11 | 31.2 ± 3.4 | 58.9 ± 11.2 |
| material | | |
| AH-Plus | 9.0 ± 1.8 | 20.3 ± 2.1 |

### EXAMPLE 13

Amine and Epoxy components, the ingredients being as follows:

| | Percent by weight |
|---|---|
| Amine component | |
| Versamid 140 | 44.0% |
| Bi₂O₃ | 49.53% |
| ZrO₂ | 3.4% |
| Aerosil R-972 | 3.0% |
| Fe₂O₃ | 0.07% |
| Epoxy component | |
| EPON 830 | 41.4% |
| EPON 862 | 5.6% |
| Bi₂O₃ | 44.6% |
| ZrO₂ | 5.4% |
| Aerosil R-972 | 3.0% |

were mixed in various volumetric ratios and allowed to cure for 24 hours at 37° C. The curing status of the samples was evaluated using a method according to ISO 6876, Dental Root Canal Sealing Materials. This procedure includes the following: a stainless steel mold 10 mm in diameter and 2 mm thick was filled with the mixture of amine and epoxy materials. After 2 minutes, the filled mold was placed into a cabinet at 37 C and 95% humidity. The mold was placed onto a metal block which had been equilibrated in the cabinet for at least 1 hour. A Gillmore-type needle with a mass of 100 g and a flat end 2.0 mm in diameter was used to periodically evaluate if the material has set. The needle was lowered onto the surface of the material and then removed. If an indentation was visible, the material was considered to be not yet fully set. As the standard protocol does not define how often the material should be evaluated, a frequency of 10 minutes was used for our testing. Testing was performed on three samples at each ratio of Epoxy to Amine and the results averaged. The results of this test are below:

| | Ratio of Epoxy Component to Amine Component | | | | |
|---|---|---|---|---|---|
| | 3:1 | 2:1 | 1:1 | 1:2 | 1:3 |
| Example | Set | Set | Set | Set | Set |
| 11 | | | | | |
| material | | | | | |
| AH-Plus | Unset | Unset | Set | Set | Set |

### EXAMPLE 14

Versamid 140 was prepared without imidazoline groups by mixing commercial Versamid 140 with 5% water and incubating at 60° C. for 48 hours to hydrolyze any imidazoline groups. The imidazoline-free Versamid 140 was mixed in a 1:1 ratio, by volume, with Epon 830 (bisphenol A diglycidyl ether) and the setting time for this mixture was measured. For comparative purposes commercial Versamid 140 was mixed in a 1:1 ratio, by volume, with Epon 830 and the setting time for this mixture was measured. The setting time of these materials was evaluated using a method according to ISO 6876 (discussed above in Example 13), Dental Root Canal Sealing Materials. It was found that the commercial Versamid 140/Epon 830 mixture cured in 4 hours and that the imidazoline-free Versamid 140/Epon 830 mixture cured in 2 hours.

Although the invention herein has been described with reference to particular embodiments, it is to be understood that these embodiments are merely illustrative of the principles and applications of the present invention. It is therefore to be understood that numerous may be made to the illustrative embodiments and that other arrangements may be devised without departing from the spirit and scope of the present invention as defined by the appended claims.

1. An epoxy polymer for dental use comprising the reaction product of a diepoxide oligomer comprised of a mixture of bisphenol A diepoxide oligomers of the structure 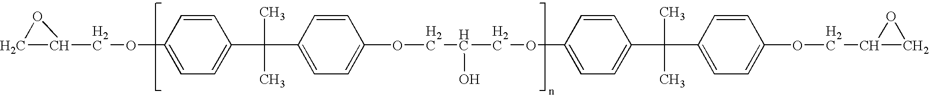
where n ranges from greater than 0 to 0.5 reacted with bridged dipolyamine monomers wherein said bridged dipolyamine monomers have two polyamine regions and a hydrocarbon region of at least 28 carbon atoms.

2. An epoxy polymer for dental use comprising the reaction product of a diepoxide oligomer comprised of a mixture of bisphenol F diepoxide oligomers of the structure 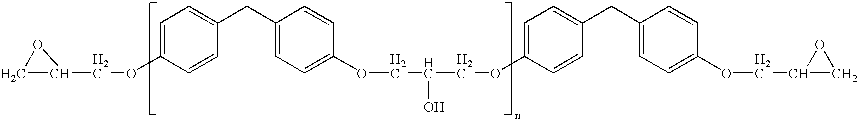
where n ranges from greater than 0 to 0.5 reacted with bridged dipolyamine monomers wherein said bridged dipolyamine monomers have two polyamine regions and a hydrocarbon region of at least 28 carbon atoms.

3. An epoxy polymer for dental use comprising the reaction product of a diepoxide oligomer comprised of a mixture of bisphenol A diepoxide oligomers of the structure 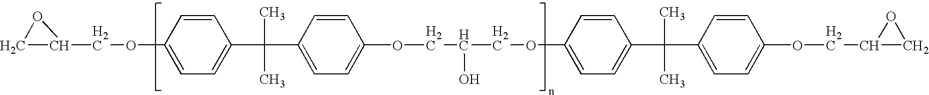
where n ranges from 0 to 0.5, and bisphenol F diepoxide oligomers of the structure 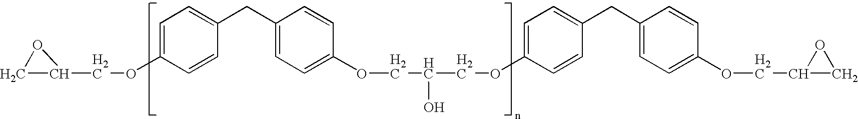
where n ranges from 0 to 0.5 reacted with bridged dipolyamine monomers wherein said bridged dipolyamine monomers have two polyamine regions and a hydrocarbon region of at least 28 carbon atoms.

4. The epoxy polymer of claims 1, 2 or 3, wherein at least some of said bridged dipolyamine monomers include a cyclized group.

5. The epoxy polymer of claim 4, wherein said cyclized group is an imidazoline.

6. The epoxy polymer of claims 1, 2 or 3, wherein said polyamine regions are selected from the group consisting of diethylene triamine, triethylene tetramine, and tetraethylene pentamine.

7. The epoxy polymer of claim 4, wherein said bridged dipolyamine monomers including a cyclized group are present in at least about 20% by weight of said bridged dipolyamine monomers.

8. The epoxy polymer of claim 7, wherein said bridged dipolyamine monomers including a cyclized group are present in at least about 50% by weight of said bridged dipolyamine monomers.

9. The epoxy polymer of claim 8, wherein said bridged dipolyamine monomers including a cyclized group are present in at least about 70% by weight of said bridged dipolyamine monomers.

10. The epoxy polymer of claim 3, wherein the ratio of said bisphenol A diepoxide oligomers to said bisphenol F diepoxide oligomers is from about 2:1 to about 20:1.

11. The epoxy polymer of claim 10, wherein the ratio of said bisphenol A diepoxide oligomers to said bisphenol F diepoxide oligomers is from about 3:1 to about 10:1.

12. The epoxy polymer of claim 11, wherein the ratio of said bisphenol A diepoxide oligomers to said bisphenol F diepoxide oligomers is about 7.4:1.

13. The epoxy polymer of claims 1 or 3, wherein said n value for said bisphenol A ranges from between 0.1 and 0.25.

14. The epoxy polymer of claim 13, wherein said n value for said bisphenol A ranges from between 0.14 and 0.2.

15. The epoxy polymer of claims 2 or 3, wherein said n value for said bisphenol F ranges from between 0.05 and 0.25.

16. The epoxy polymer of claim 15, wherein said n value for said bisphenol F ranges from between 0.07 and 0.13.

17. The epoxy polymer of claims 1, 2 or 3, wherein said hydrocarbon region includes at least 32 carbon atoms.

18. The epoxy polymer of claim 17, wherein said hydrocarbon region includes at least 36 carbon atoms.

19. The epoxy polymer of claims 1, 2 or 3, wherein said hydrocarbon containing region is derived from the dimer of a fatty acid or fatty alcohol.

20. The epoxy polymer of claims 1, 2 or 3, wherein said diepoxide oligomer is present in an amount of about 3:1 to about 1:3 by weight relative to the amount of said bridged dipolyamine monomers.

21. The epoxy polymer of claim 20, wherein said diepoxide oligomer is present in an amount of about 1.5:1 to about 1:1.5 by weight relative to the amount of said bridged dipolyamine monomers.

22. The epoxy polymer of claim 21, wherein said diepoxide oligomer is present in an amount of about 1:1 by weight relative to the amount of said bridged dipolyamine monomers.

23. The epoxy polymer of claims 1, 2 or 3, further comprising a filler.

24. An epoxy polymer for dental use comprising the reaction product of a diepoxide oligomer comprised of a mixture of bisphenol A diepoxide oligomers of the structure 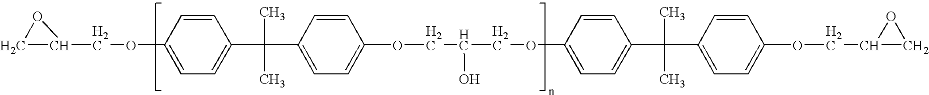
where n ranges from between 0 and 0.5, and bisphenol F diepoxide oligomers of the structure 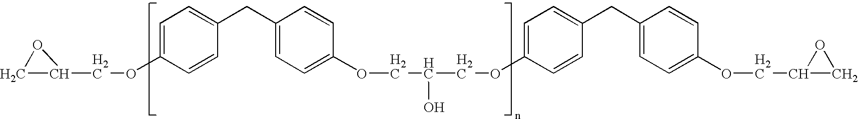
where n ranges from between 0 and 0.5, and wherein the ratio of said bisphenol A diepoxide oligomers to said bisphenol F diepoxide oligomers is from about 2:1 to about 20:1; and a plurality of bridged dipolyamine monomers having a hydrocarbon region and two polyamine regions, at least one bridged dipolyamine monomer including an amine region having a cyclized group and wherein said bridged dipolyamine monomer including said cyclized group is present in at least about 20% by weight of said plurality of bridged dipolyamine monomers, and wherein said hydrocarbon region has at least 28 carbon atoms, and wherein said diepoxide oligomer is present in an amount of about 3:1 to about 1:3 by weight relative to the amount of said bridged dipolyamine monomers.
